(19) United States
(12) Patent Application Publication
(10) Pub. No.: US 2014/0180289 A1
(43) Pub. Date: Jun. 26, 2014

(54) Femur Supporting Device

(71) Applicant: METAL INDUSTRIES RESEARCH & DEVELOPMENT CENTRE, Kaohsiung City (TW)

(72) Inventors: Kuang-Sheng Lee, Kaohsiung City (TW); Pei-Yuan Lee, Kaohsiung City (TW); Wei-Jen Shih, Kaohsiung City (TW); Ming-Hsien Hu, Kaohsiung City (TW); Yen-Nien Chen, Kaohsiung City (TW)

(21) Appl. No.: 13/913,592

(22) Filed: Jun. 10, 2013

(30) Foreign Application Priority Data
Dec. 25, 2012 (TW) 101149826

(51) Int. Cl. A61B 17/74

(52) U.S. Cl. CPC A61B 17/744
USPC 606/64

(57) ABSTRACT

A femur supporting device includes a rod having an insertion groove and a fixing through-hole. Each of the insertion groove and the fixing through-hole extends through an outer periphery of the rod. An insert is engaged in the insertion groove of the rod. A first fastener is engaged with the fixing through-hole of the rod. The femur supporting device provides enhanced stability.

### BACKGROUND OF THE INVENTION

1. Field of the Invention

The present invention relates to a femur supporting device and, more particularly, to a femur supporting device mounted in a femur for cooperating with an artificial femoral head to form an artificial hip joint.

2. Description of the Related Art

With reference to FIGS. 1 and 2, Taiwan Invention Patent Publication No. I312677 entitled “ARTIFICIAL FEMUR STRUCTURE” discloses a femur supporting device 9 including a seat 91 and a stem 92. The seat 91 includes a conical through-hole 911 having a plurality of positioning grooves 912 in an inner periphery thereof. The stem 92 includes a bone shaft end 921 and a joint end 922. The bone shaft end 921 includes a conical section 923 having a key 924. The stem 92 is extended through the through-hole 911 of the seat 91, and the conical section 923 is in tight coupling with the through-hole 911. The key 924 is engaged in one of the positioning grooves 912 to fix the angular position of the joint end 922 of the stem 92 while reliably positioning the stem 92.

When the conventional femur supporting device 9 is implanted into a human body, the muscles and ligaments covering the femur F of the human body are incised, and the femoral neck and the femoral head are cut off. Next, the cut surface of the femur is ground to a flat surface, and a tool is used to dig a hole in the flat cut surface. The outline of the femur supporting device 9 damages the cancellous bone in the femur F to obtain a space for receiving the femur supporting device 9. Then, the femur supporting device 9 is placed into the space and enters the medullary cavity of the femur F. Bone cement is filled into the gaps in the medullary cavity not filled by the femur supporting device 9. Implantation of the femur supporting device 9 is, thus, completed.

Since the joint end 922 of the stem 92 is obliquely and integrally connected to the bone shaft end 921 of the stem 92, the linea intertrochanterica between the trochanter major and the trochanter minor has to be removed in order to smoothly implant the femur supporting device 9 into the femur F. Thus, most undamaged bone tissues on the upper end of the femur F are removed. This requires a wider operative field, and the operation wound can not be reduced. Thus, the operation time is long, the operation risks are high, and the patient needs a longer recovery time after surgery.

Furthermore, a period of time is required for the bone cement in the gaps to harden. If the femur supporting device 9 is moved before the bone cement completely hardens, voids are liable to be formed, and the femur supporting device 9 can not provide the expected supporting effect. Further, the bone cement can not provide the desired fixing effect for a patent suffering serious osteoporosis, leading to a toxic hazard resulting from incomplete aggregation of the bone cement. Further, since the elastic modulus of the bone cement is smaller than the bones and since the shear strength of the bone cement is smaller than the pressure resistance, the femur supporting device 9 implanted in the femur F can not provide an anti-torque effect. As a result, it is not uncommon that the femur supporting device 9 malfunctions after a period of time.

Taiwan Utility Publication No. M394805 entitled “FEMUR PROSTHESIS”, Taiwan Utility Publication No. M389530 entitled “HIGH PRESSURE RESISTANT ARTIFICIAL HIP JOINT FILLED WITH ANTIBIOTIC BONE CEMENT”, and Taiwan Invention Patent No. I305722 entitled “ARTIFICIAL BONE WITH POROUS TISSUES” discloses structures similar to the conventional femur supporting device 9 and, thus, have the above disadvantages. Thus, improvement to the conventional femur supporting devices is required.

### SUMMARY OF THE INVENTION

An objective of the present invention is to provide a femur supporting device that can save most of the bone tissues and that can be implanted into a femur through a minimally invasive surgery, saving the operation time, reducing the operation risks, and reducing the aftereffects.

Another objective of the present invention is to provide a femur supporting device reliably engages with the femur while providing an anti-torque effect.

A further objective of the present invention is to provide a femur supporting device fixed by engagement and locking without using bone cement.

The present invention fulfills the above objective by providing a femur supporting device including a rod having an insertion groove and a fixing through-hole. Each of the insertion groove and the fixing through-hole extends through an outer periphery of the rod. An insert is engaged in the insertion groove of the rod. A first fastener is engaged with the fixing through-hole of the rod.

Preferably, the rod includes a head end and an implant end. The insertion groove is located adjacent to the head end of the rod, and the fixing through-hole is located adjacent to the implant end of the rod.

Preferably, the insertion groove obliquely extends through the rod.

Preferably, the insertion groove includes an inlet and an outlet aligned with the inlet. The insert extends through the inlet and the outlet of the insertion groove. The insertion groove has decreasing cross sectional widths from the inlet towards the outlet.

Preferably, the insert includes first and second ends. The insert further includes an insertion section and an exposed section between the first and second ends. The insert section is located between the first end and the exposed section. The exposed section is located between the second end and the insert section. The first end of the insert is extended through the insertion groove of the rod. The insertion section of the insert is engaged in the insertion groove of the rod.

Preferably, the insertion section of the insert has decreasing cross sectional widths towards the first end of the insert. The maximum cross sectional width of the insertion section of the insert is larger than the cross sectional width of the inlet of the insertion groove.

Preferably, the insert further includes a locking hole in an end face of the first end. A second fastener is engaged in the locking hole of the insert.

Preferably, the exposed section of the insert includes a ball head connecting portion in a form of a cylinder. The ball head connecting portion has a diameter smaller than the maximum cross sectional width of the insertion section adjacent to the exposed section.

Preferably, the exposed section of the insert includes an abutment protrusion formed on an outer periphery of the insert at the second end of the insert.

The first fastener is adapted to extend from a side of a femur through another side of the femur. The first fastener is extended through and engaged with the fixing through-hole of the rod. The abutment protrusion is adapted to abut a cut end edge of a femur neck.

By using the rod longitudinally implanted into the medullary cavity of the femur and the insert obliquely inserted through the rod, only the neck of the femur is cut in the surgery. Most of the bone tissues of the femur can be kept, and the rod and the insert can be implanted without trimming the outline of the femur. The operative field and the operation wound are small (namely, the surgery is a minimally invasive surgery), significantly saving the operation time and reducing the operation risks and aftereffects.

By using the fastener penetrating through the cortical bone of the femur to provide a positioning effect relative to the rod and by the other fastener extended into the insert from the outer edge of the femur and engaged with the insert, the insert and the femur can not move relative to each other, enhancing the engaging reliability between the insert, the rod, and the femur. Thus, the femur supporting device provides enhanced stability and enhanced anti-torque effect and, thus, provides excellent supporting effect and is durable.

By engaging the insert with the rod and by fixing the rod and the insert to the femur by the fasteners, the femur supporting device can be implanted into and fixed in the femur through a simple mechanical structure without using bone cement, avoiding disadvantages and side effects resulting from the bone cement.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 shows a cross sectional view of a conventional femur supporting device.

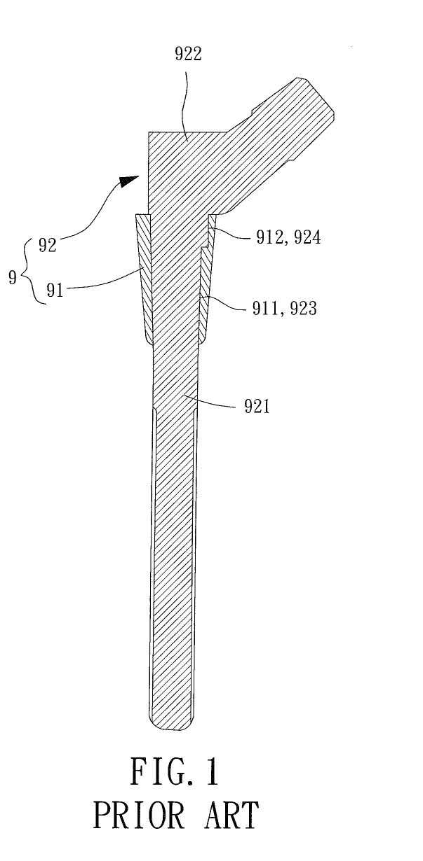

FIG. 2 shows a cross sectional view illustrating the use of the conventional femur supporting device.

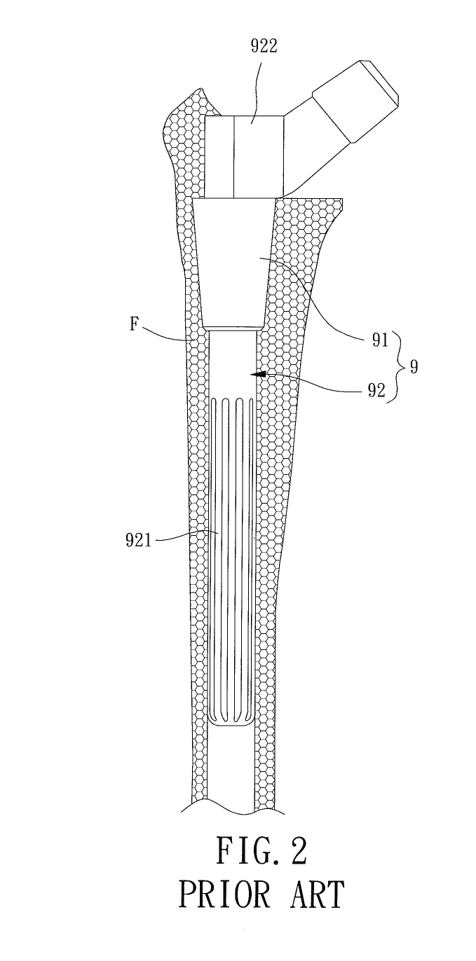

FIG. 3 shows an exploded, perspective view of a femur supporting device according to the present invention.

FIG. 4 shows a schematic view illustrating the use of the femur supporting device according to the present invention.

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

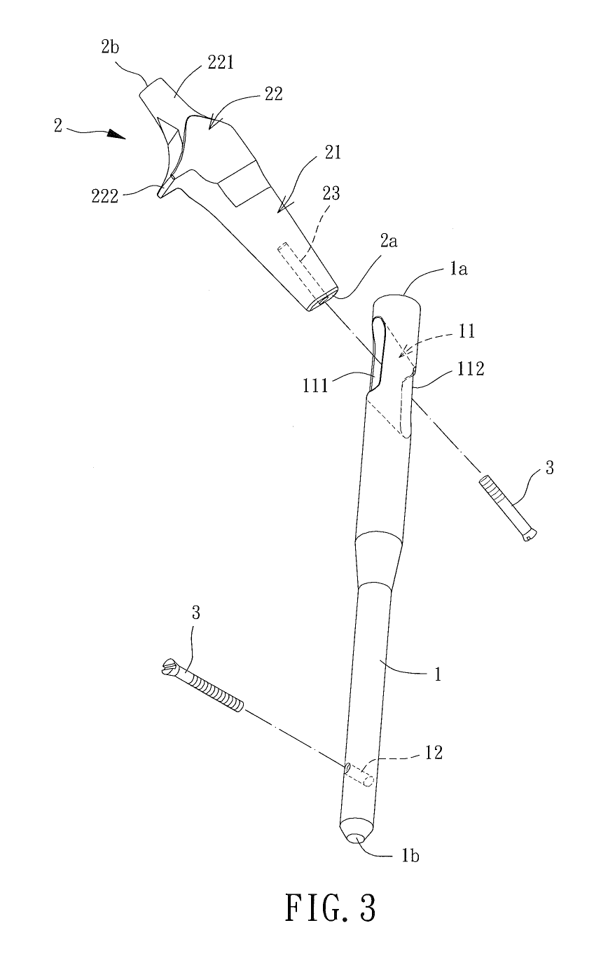

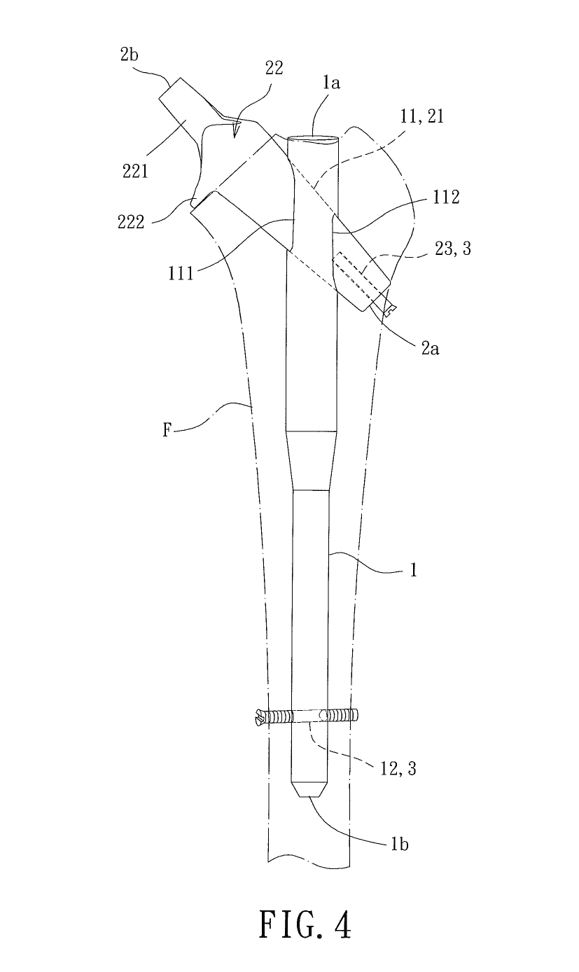

With reference to FIGS. 3 and 4, a femur supporting device according to the present invention includes a rod 1, an insert 2, and a fastener 3. The rod 1 can be longitudinally implanted into a medullary cavity of a femur F of a patient. The insert 2 is engaged with the rod 1 and is adapted to couple with an artificial femoral head. The fastener 3 extends from a side of the femur F through the other side of the femur F to fix the rod 1.

The rod 1 includes a head end 1a and an implant end 1b spaced from the head end 1a along a longitudinal axis. The rod 1 is implanted into the medullary cavity of the femur F through the implant end 1b. The rod 1 includes an insertion groove 11 extending through an outer periphery of the rod 1 and having an inlet 111 and an outlet 112 aligned with the inlet 111, allowing insertion and engagement of the insert 2. In this embodiment, the insertion groove 11 has decreasing cross sectional widths from the inlet 111 towards the outlet 112 to securely engage with the insert 2.

Preferably, the insertion groove 11 is located adjacent to the head end 1a of the rod 1. Furthermore, the insertion groove 11 obliquely extends through the rod 1 to match the angular position of the original neck of the femur F relative to the medullary cavity. Preferably, the insertion groove 11 is at an acute angle in a range between 30°-60° to the longitudinal axis of the rod 1. The rod 1 further includes a fixing through-hole 12 extending through the outer periphery of the rod 1. The fixing through-hole 12 is located adjacent to the implant end 1b of the rod 1 to provide enhanced anti-torque effect after implantation. It can be appreciated that more than one fixing through-hole 12 and more than one corresponding fastener 3 can be used.

The insert 2 includes first and second ends 2a and 2b. The insert 2 further includes an insertion section 21 and an exposed section 22 between the first and second ends 2a and 2b. The insertion section 21 is located between the first end 2a and the exposed section 22. The exposed section 22 is located between the second end 2b and the insertion section 21. After implantation, the first end 2a and the insertion section 21 are located in the medullary cavity of the femur F, and the exposed section 22 and the second end 2b are exposed at the cut neck of the femur F.

The first end 2a of the insert 2 is extended through the insertion groove 11 of the rod 1. The insertion section 21 of the insert 2 is engaged in the insertion groove 11 of the rod 1. Thus, the insert 2 is securely engaged with the rod 1. In this embodiment, the insertion section 21 of the insert 2 has decreasing cross sectional widths towards the first end 2a. The maximum cross sectional width of the insertion section 21 of the insert 2 is larger than the cross sectional width of the inlet 111 of the insertion groove 11. Thus, when the insert 2 is inserted into the rod 1, the inlet 111 of the insertion groove 11 restricts the insertion section 21 of the insert 2 to prevent the whole insert 2 from extending through the outlet 112 of the insertion groove 11. Furthermore, the insert 2 includes a locking hole 23 in an end face of the first end 2a for engagement with another fastener 3.

The exposed section 22 of the insert 2 includes a ball head connecting portion 221 to replace the cut neck of the femur F for coupling with an artificial femoral head. The ball head connecting portion 221 has an outline corresponding to the artificial femoral head. In this embodiment, the ball head connecting portion 221 is in a form of a cylinder. Preferably, the ball head connecting portion 221 has a diameter smaller than the maximum cross sectional width of the insertion section 21 adjacent to the exposed section 22. Furthermore, the exposed section 22 of the insert 2 includes an abutment protrusion 222 formed on an outer periphery of the insert 2 at the second end 2b.

With reference to FIG. 4, in use of the femur supporting device, the neck of the femur F of the patient is cut, and the rod 1 is placed into the medullary cavity of the femur F by the implant end 1b. The first end 2a of the insert 2 is extended through the insertion groove 11 of the rod 1. The inlet 111 of the insertion groove 11 provides a positioning effect for the insertion section 21 of the insert 2 while imparting a longitudinal pressure to the insert 2 such that the insertion section 21 is in tight coupling with the insertion groove 11, securely engaging the insert 2 with the rod 1. Furthermore, the abutment protrusion 222 abuts the cut end edge of the neck of the femur F. The fastener 3 is extended from a side of the femur F through other side of the femur F and is extended through and engaged with the fixing through-hole 12 of the rod 1. The other fastener 3 is mounted into and engaged with the locking hole 23 of the insert 2 from an outer edge of the femur F, preventing undesired displacement between the insert 2 and the femur F and enhancing the engaging reliability between the insert 2, the rod 1, and the femur F. After the femur supporting device is implanted into the femur F, an artificial femoral head (such as a ceramic joint head) corresponding to the ball head connecting portion 221 is mounted to the ball head connecting portion 221, forming an artificial hip joint and restoring normal functions of the joint.

Since the abutment protrusion 222 of the femur supporting device abuts the cut end edge of the neck of the femur F, the force acting on the hip joint can be imparted to the cortical bone and withstood by stronger portions of the femur F, reducing the force imparted to the portions of the rod 1 and the insert 2 implanted in the medullary cavity of the femur F. Thus, the femur F of the patient is less likely to break.

In the femur supporting device according to the present invention, by using the rod 1 longitudinally implanted into the medullary cavity of the femur F and the insert 2 obliquely inserted through the rod 1, only the neck of the femur F is cut in the surgery. Most of the bone tissues of the femur F can be kept, and the rod 1 and the insert 2 can be implanted without trimming the outline of the femur F. The operative field and the operation wound are small (namely, the surgery is a minimally invasive surgery), significantly saving the operation time and reducing the operation risks and aftereffects.

In the femur supporting device according to the present invention, by using the fastener 3 penetrating through the cortical bone of the femur F to provide a positioning effect relative to the rod 1 and by the other fastener 3 extended into the insert 2 from the outer edge of the femur F and engaged with the insert 2, the insert 2 and the femur F can not move relative to each other, enhancing the engaging reliability between the insert 2, the rod 1, and the femur F. Thus, the femur supporting device provides enhanced stability and enhanced anti-torque effect and, thus, provides excellent supporting effect and is durable.

In the femur supporting device according to the present invention, by engaging the insert 2 with the rod 1 and by fixing the rod 1 and the insert 2 to the femur F by the fasteners 3, the femur supporting device can be implanted into and fixed in the femur F through a simple mechanical structure without using bone cement, avoiding disadvantages and side effects resulting from the bone cement.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A femur supporting device comprising:
a rod including an insertion groove and a fixing through-hole, with each of the insertion groove and the fixing through-hole extending through an outer periphery of the rod;
an insert engaged in the insertion groove of the rod; and
a first fastener engaged with the fixing through-hole of the rod.

2. The femur supporting device as claimed in claim 1, with the rod including a head end and an implant end, with the insertion groove located adjacent to the head end of the rod, with the fixing through-hole located adjacent to the implant end of the rod.

3. The femur supporting device as claimed in claim 1, with the insertion groove obliquely extending through the rod.

4. The femur supporting device as claimed in claim 1, with the insertion groove including an inlet and an outlet aligned with the inlet, with the insert extending through the inlet and the outlet of the insertion groove, with the insertion groove having decreasing cross sectional widths from the inlet towards the outlet.

5. The femur supporting device as claimed in claim 4, with the insert including first and second ends, with the insert further including an insertion section and an exposed section between the first and second ends, with the insert section located between the first end and the exposed section, with the exposed section located between the second end and the insert section, with the first end of the insert extended through the insertion groove of the rod, with the insertion section of the insert engaged in the insertion groove of the rod.

6. The femur supporting device as claimed in claim 5, with the insertion section of the insert having decreasing cross sectional widths towards the first end of the insert, with a maximum cross sectional width of the insertion section of the insert larger than the cross sectional width of the inlet of the insertion groove.

7. The femur supporting device as claimed in claim 5, further comprising: a second fastener, with the insert further including a locking hole in an end face of the first end, with the second fastener engaged in the locking hole of the insert.

8. The femur supporting device as claimed in claim 5, with the exposed section of the insert including a ball head connecting portion in a form of a cylinder, with the ball head connecting portion having a diameter smaller than the maximum cross sectional width of the insertion section adjacent to the exposed section.

9. The femur supporting device as claimed in claim 5, with the exposed section of the insert including an abutment protrusion formed on an outer periphery of the insert at the second end of the insert.

10. The femur supporting device as claimed in claim 9, with the first fastener adapted to extend from a side of a femur through another side of the femur, with the first fastener extending through and engaged with the fixing through-hole of the rod, with the abutment protrusion adapted to abut a cut end edge of a femur neck.
